(19) United States
(12) Patent Application Publication
(10) Pub. No.: US 2012/0295299 A1
(43) Pub. Date: Nov. 22, 2012

(54) Method and apparatus for rapidly analyzing microorganisms using petri plates

(76) Inventor: Sergey Gazenko, Mason, OH (US)

(21) Appl. No.: 13/068,646

(22) Filed: May 17, 2011

(51) Int. Cl. C12Q 1/04; C12M 1/34

(52) U.S. Cl. 435/34; 435/287.1

(57) ABSTRACT

A growth plate or Petri plate and a method for detection and identification of microorganisms by specific or non-specific staining of colonies or microcolonies is described. Analytical substances (chromogenic or fluorogenic substrates, biochemical dyes, dyes indicators of pH, antibiotics and other biological active substances) are applied to the lower surface of a nutrient agar layer. The substances diffuse in the agar, reach cells and react with them. Because colonies or microcolonies are not removed from the media, they retain their shape, color, size and other useful characteristics for analytical purposes.

### BACKGROUND OF THE INVENTION

The present invention relates generally to the field of microbiology and in particular to the field of rapidly detecting, identifying, and enumerating microorganism colonies and micro-colonies.

Growth on Petri plates is a typical microbiological operation used in medical, industrial, biotechnological, research, and pharmaceutical laboratories. Hundreds of millions of analyses are performed worldwide by these methods each year.

The Petri plate, also known as a Petri dish or culture dish is commonly found in laboratories around the world and has been in use for many decades. The main purpose of the Petri plate in biologically oriented laboratories is growing clones from one cell to create larger quantities of cells for analysis. The analyses include activities such as counting live cells in colonies that appear after a period of preliminary growth to establish the number of Total Viable Organisms (TVO) or such as differentiation by colony shape or by colony color. Also identification by other methods such as classical biochemical analysis, chromatography of fatty acids, Enzyme-Linked Immunosorbent Assay (ELISA), mass-spectrometry, Fourier Transform InfraRed (FTIR) spectroscopy, or immuno-analyses can be accomplished. Despite the fact that detection and identification of cells, in many cases, can be done by modern techniques such as flow cytometry or Polymerase Chain Reaction (PCR), which are complex and expensive methods, growing a pure culture that is a colony from one live cell, still dominates laboratory practice.

Nutrient agars are nearly always used for growing these colonies. Usually colonies containing clones of one initial cell are removed from the microbiological growth medium, usually nutrient agar, and are further analyzed using the above mentioned methods. However, there are two methods of identification which do not require removal of colonies from the nutrient agar. One of these methods is related to the microbiological agar media marketed under the trademark CHROMAGAR® and colloquially known by the name CHROMAGAR®, implying that microorganisms grown on these agar media do not require removal for analysis and the other method is known as permeable membranes. Both methods allow specific or non-specific staining of colonies for analytical purposes without removing the colonies from the Petri plate.

The CHROMAGAR® microbiological growth media or nutrient media contain special substances, substrates, and antibiotics that allow growth and simultaneous specific coloration of colonies of interest. The CHROMAGAR® Candida, CHROMAGAR® 0157, CHROMAGAR® Salmonella, CHROMAGAR® Aureus as well as some other CHROMAGAR® microbiological agar media are currently used for identifying colonies of interest by color (pink, green, or blue).

The substances initiating coloration get collected in the cellular bodies causing growth problems. Therefore, colonies are atypically small and very often need prolonged incubation. Only regular-sized colonies can be detected by this method because the colors tend to be weak, and microcolonies with poor light absorption are ineffective for microscopic identification. Also, only a limited number of species can grow on CHROMAGAR® microbiological growth media. Therefore, the CHROMAGAR® media are not useful for the comprehensive TVO microbial detection and enumeration that is most commonly used in microbiology.

The other method that allows analysis without removal is the method of permeable membranes used for rapidly detecting microcolonies. In this method the Petri plate contains a permeable membrane on the surface of a nutrient medium with a thin layer of the same nutrient medium placed on the membrane. After ¼-⅓ of the usual growth time, which is usually 24, 48 or 72 hours, the membrane having small invisible microcolonies is removed to another plate containing a chromogenic substrate which stains the cells and makes them easily visible. Staining reveals the shape of the microcolonies which can be seen using a microscope and which can be helpful for preliminary identification or differentiation.

However, installation of the permeable membrane on the microbiological growth medium is a complicated, time consuming, manual process. The method requires a secondary plate filled with agar containing chromogen. Mounting a membrane on a secondary plate (with chromogen) may lead to the formation of liquid pads that wash out microcolonies and prevent their counting and shape recognition.

Only a few chromogens can be stored for any length of time on a secondary plate. This method is designed exclusively for the detection of microcolonies. Hence, staining regular colonies is redundant. Generally, the method of permeable membranes is attuned for only one chromogen—Methyltyazolyl Tetrazolium Bromide (MTT).

Ideally, one would like to do these analyses without needing to use a permeable membrane. Further, it would be desirable to effectively use any combination of substances in the analysis of both microcolonies and regular colonies and, ideally, require only one plate for the analysis.

The combination of a new method together with a new design for a Petri plate which will provide significantly shortened time for analysis and simplified procedures while saving material and financial resources could lead to improved microbiological analysis providing a needed positive effect on medical diagnostics and industrial and environmental analysis.

### BRIEF DESCRIPTION OF THE INVENTION

An apparatus for growing microorganisms is provided. The apparatus comprises a container having at least one wall with a substantially open top side and a substantially open bottom side. A top cover for covering the top side of the container to prevent contamination of the microorganisms within the container along with a microbiological growth medium and a structure for supporting the medium is also provided.

A removable bottom cover is provided that allows access to the bottom side of the growth medium through the open bottom of the container. With the bottom cover removed access to the bottom side of the growth medium is made available allowing analytical substances to be placed in contact with the growth medium and in turn allowing the analytical substances to pass through the growth medium and affect the microorganisms disposed on the opposite side of the growth medium.

A method for detecting and identifying microorganisms is also provided. The method comprises a series of steps.

A container for growing the microorganisms having at least one wall, a substantially open top side, a substantially open bottom side, a top cover, a structure for supporting a microbiological growth medium, and a removable bottom cover is prepared.

Colonies of cells are grown on the upper surface of the microbiological growth medium in the container.

With the bottom cover removed contact with the lower surface of the growth medium is made with a substance capable of passing through the growth medium.

The microorganisms are incubated and molecules of the substance pass through the growth medium reaching the colonies and reacting with them.

The reaction between the microorganisms and the substance capable of passing through the growth medium is detected and the results are analyzed.

### BRIEF DESCRIPTION OF THE FIGURES

FIG. 1 is a Petri plate.

FIG. 2 is a container with a growth medium inside.

FIG. 3 A is a container with growth medium, microorganism colonies and analytical substance

FIG. 3 B is a container with analytical substance reaching microorganisms.

FIG. 4 A is Petri plates with no visible microcolonies.

FIG. 4 B is Petri plates with microcolonies visible.

FIG. 5 is different sizes and colors of microorganism colonies.

FIG. 6 is microorganisms stained by chromogenic substrates.

FIG. 7 is stained microcolonies after several hours of incubation.

### DETAILED DESCRIPTION OF THE INVENTION

Petri plates come in many different sizes and shapes, such as round or rectangular. For example, 35 mm, 60 mm, 95 mm and other circular diameters are common. Various depths are also available. The plates can contain pads inside or a grid on the bottom for convenient counting. Some plates have two, three or more compartments.

Another type of Petri plate known as a contact plate consists of a raised agar bed for collecting microbes from surfaces. Petri plates are usually made of glass for reusability or polymer, often polystyrene, for disposability.

However, Petri plates without a bottom that allow access to the lower surface of the nutrient agar have not been available. Methods for adding analytical substances to live cells and transporting the substances through an agar layer are also unknown. The addition of substances that are able to pass through the agar layer more or less rapidly and react with live cells disposed on the opposite side of the agar layer have several significant advantages and are discussed in what follows.

The invention of the Open Bottom Side (OBS) Petri plate obviates the need for a permeable membrane for colony removal. Any combination of substances can be applied to the bottom surface. OBS plates can be used for microcolonies as well as regular colonies. Also, the OBS plate needs only half the amount of agar and only one plate for analysis. Analytical opportunities provided by the OBS plate are much broader because many different substances such as chromogens and dyes can be used for analysis.

The following terms and explanations are incorporated herein to define and further elucidate the discussion of the invention

Nutrient medium: Nutrient medium is a growth medium used for growing colonies or microcolonies within the context of the current invention. A nutrient medium can be a general medium for promoting bacterial growth such as Tryptic Soy Agar, a medium for fungi and yeast growth such as Saboraud Dextrose Agar, or a medium used for growth of special groups of microorganisms, such as Cetrimide Agar used for growth of Pseudomonas species, or MacConkey Agar used for the revealing of Gram-negative enteric pathogens.

Microcolony: A microcolony is a small colony of cells appearing after 3-6 hours of incubation. The typical size of microcolonies is 10-100 μm. They are colorless and invisible in a regular light microscope. Even colored microcolonies like yellow Staphylococcus (Staph.) aureus, are not usually visible in a light microscope. Therefore, microcolonies typically need to be colored to be visible. The majority of microbial species produce microcolonies of specific shape during early stages of growth (3-6 hours). Quite often, differences in their shape are so evident that analysts can differentiate species simply by the shape of a given microcolony. Microcolonies consist of chains of cells which are not mechanically connected to each other. Therefore, all growth media and secondary media must not contain free liquids, lest the cells of the microcolonies dissociate from each other. All nutrient liquids and dissolved substances are bound by agarose or another polymeric gel.

Removable element: A removable element serves to keep nutrient agar intact and sterile during Petri plate preparation and during incubation with the sample spread on the surface of the nutrient medium. The removable element can be a polymeric autoclavable membrane that is non-permeable for liquids. Glue on the edges of membrane can be helpful for sticking it to lower part of container for nutrient agar. Other examples for materials comprising the removable element can be metals such as aluminum or another foil, glass, non-permeable paper or thin rigid plastic/polymer. Another shape for the removable element can be a plate that is applied to the bottom of the container by a contact clip or forced attachment or by threading the containers together.

Container for nutrient agar: Such a container is the main element for accomplishing the invention. The container has no bottom or has a substantially open bottom side through which to apply analytical substances under the nutrient agar.

Analytical substances: Analytical substances are indicators and substances that are able to specifically or non-specifically label colonies or microcolonies and the space around them or reveal influence on their growth, shape or size. Often they are chromogenic and/or fluorogenic substrates or their mixtures, labeled or regular antibodies, enzymes, detergents or other biologically active substances. Small molecules of these substrates can freely pass through the gel discussed below, and react with enzymes, membranes, antigens and other structures of live cells. Some examples of these substances are: Tetrazolium Blue Chloride (TBC) available from Sigma-Aldrich T4375, Iodonitrotetrazolium Chloride (INT) available from Sigma-Aldrich 18377, Nitrotetrazolium Blue Chloride (NBC) available from Sigma-Aldrich N6639, Methyltyazolyl Tetrazolium Bromide (MIT) available from Sigma-Aldrich M2128, and Oxacillin sodium salt available from Sigma-Aldrich 28221.

Detection, differentiation, and identification: The term “detection” is generally understood to mean the ability to reveal and count/enumerate any colonies or microcolonies independent of their taxonomic classifications. “Differentiation” means the ability of a method to differentiate two or more species from each other by their shape, color, or wavelength/color of fluorescence and other features. “Identification” means the determination of genus and species of a given colony or microcolony.

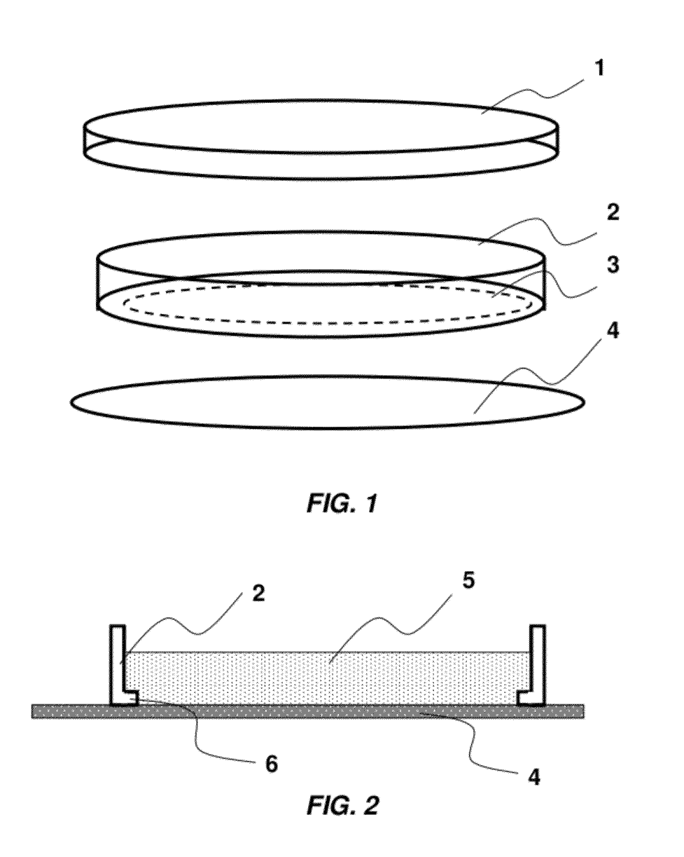

One embodiment of a Petri or growth plate is shown in FIG. 1. The plate consists of three parts: the top side cover or lid 1, the growth medium container 2 with a substantially open bottom side 3 and a removable membrane 4. Top side cover 1 is the same as that found on a common Petri plate. The container for the growth medium 2 has a substantially open bottom side 3 for contact with analytical substances after the growth medium such as nutrient agar is solidified on the inner surface of removable membrane 4. The functions of the removable membrane are 1) holding liquid nutrient agar during solidification and 2) protecting the agar from contamination during storage. In this embodiment the membrane is removed only after incubation of the sample to place analytical substances adjacent to or in contact with the bottom side of the growth medium, the bottom side being the side opposite the top side on which the growing organisms are disposed.

In another embodiment a slightly differently shaped container can be used for the growth medium. This container provides a supporting structure for better holding the agar in place and for protecting the agar from falling out of the container during the manipulations that occur during processing. Such a structure could comprise a permeable plastic or another inert material that is cut in thin strips or could comprise strips and rings of the material. Such a structure could be placed in the plate before pouring the liquid growth medium thereon or could even be part of the container 2.

Another embodiment provides convex or concave shapes or ridges on a wall of container 2 in the area it contacts the growth medium, which is usually nutrient agar. These bulging or concave shapes or ridges are useful for keeping the growth medium immobilized and confined in the container 2.

FIG. 2 shows the growth medium container 2, containing the growth medium 5 protected by a removable membrane 4. The removable membrane 4 can replace the bottom cover of the Petri plate. The membrane 4 can be attached to the bottom of the Petri plate in different ways.

For example, in one embodiment the edge of the membrane 4 can be coated with adhesive around its edge to hold the membrane 4 against the narrow ridge 6 as shown in FIG. 2.

In another embodiment the membrane 4 can be secured to the bottom of the open bottom Petri dish by applying a tightly fitting ring around the bottom of the container 2 and securing the membrane to the bottom of the dish by folding it up on the side of the container so that it is frictionally held between the side of the container 2 and the ring.

In this embodiment the ring could also be the side of a bottom cover that is similar to an inverted top cover but is tightly fitted to provide a friction fit to the container side 2. In this embodiment, where the bottom cover is constructed similarly to an inverted top cover 1, the bottom cover can also support the membrane 4 as the membrane lies on the surface comprising the inside surface of the bottom cover. In some cases such support could allow the membrane 4 to be loaded more heavily than would be allowed without the support of the closed bottom cover and analytical materials can be placed in the closed bottom for diffusion through the membrane.

Another embodiment allows the removable membrane to comprise a part of a friction fit ring wherein the membrane is a part of a structure that can be friction fit to the bottom of the container 2.

In another embodiment, a threaded connection between the container side 2 and the bottom membrane 4 holding device can be used to secure the membrane 4 in place. This can be accomplished by incorporating threads on the outside of the container 2 so that a threaded membrane supporting ring can be secured to the bottom of the Petri plate.

In yet another embodiment when the apparatus is used, a sample containing cells is placed in the Petri plate on the surface of the growth medium and is incubated for a certain time and temperature. Penetration by liquids through the agar incorporated in the sample or placed on top of the sample can take some time and can cause errors in analysis if the penetration time is too long. When this happens, membrane 4 can be temporarily removed and a sterile pad or blotting paper can be attached to the bottom of the growth medium. This simple procedure draws liquids from the sample in a matter of minutes. This is another useful feature of the open-bottom container. After the upper surface of the growth medium becomes dry, one can replace the membrane 4 and continue processing the sample.

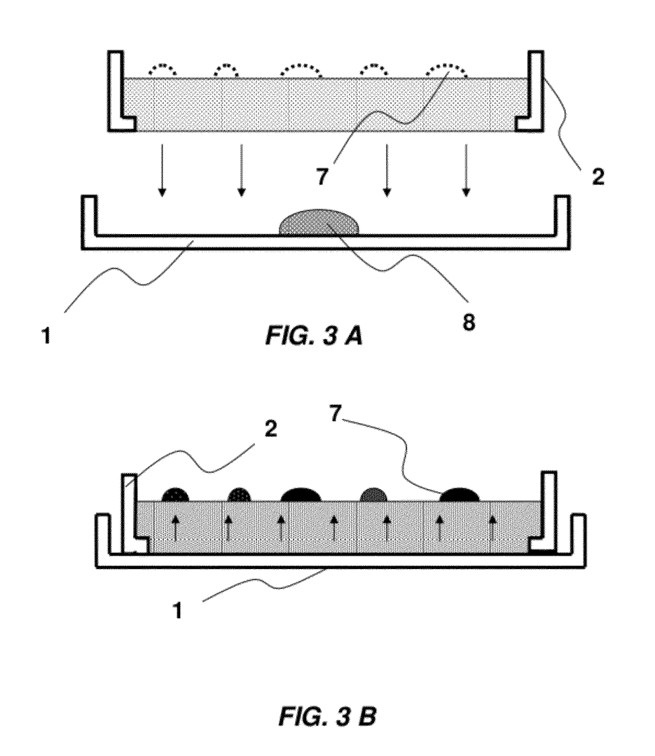

From another aspect, referring to FIG. 3, after the needed incubation time the membrane 4 is removed and the container 2 with the growth medium and colonies or microcolonies 7 is placed in contact with a drop 8 containing analytical substances as shown in FIG. 3 A. The drop 8 can be placed in the inverted top side lid of Petri plate 1 or another container that is inert with regard to the analytical substances. Examples of these could be plastic, glass, or foil. Experiments show that for a normal size Petri plate, a 0.8-1.2 ml drop is enough to spread over the lower surface of nutrient agar filled plate. After the container 2 with the growth medium 5 and colonies 7 is placed on a drop of analytical substances, the substances immediately start penetrating the growth medium as shown in FIG. 3B.

Gels, such as agar, mainly consist of liquid but behave like solid substances. For example, agar is stabilized by the presence of water molecules bound inside polymer cavities. These cavities are connected to each other allowing molecules of water and other substances to move by diffusion inside the agar. Molecules will spread inside agar's cross-linked network in the same manner as in a liquid but more slowly. Other gels such as carrageenan and gelatin can also be used as they have the same properties.

Molecules of analytical substances spread inside the growth medium 5 (FIG. 3 B) depending on concentration or density, molecular weight, and temperature of the growth medium. For example, experiments show that molecules of agar having molecular weight 300-800, at temperatures of 35-40° C. reach the upper surface of nutrient agar having a thickness of 4-5 mm in 30-40 minutes. At room temperature such movement takes 0.75-1.2 hour. FIGS. 3 A and B demonstrate the color change in colonies on application of a chromogenic substrate to the lower surface of growth medium nutrient agar.

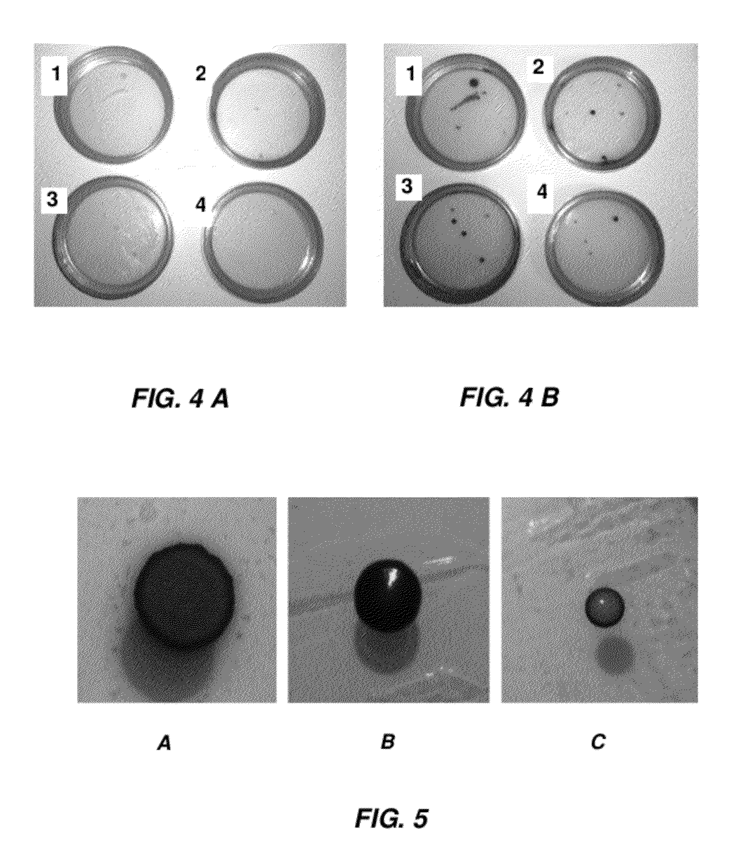

FIGS. 4 A and B demonstrate rapid staining of a mixture of species: E. coli O157:H7, Staphyllococcus aureus and Bacillus brevis. Plates A1, A2, B1 and B2 are stained by mixture of TBC, INT and MTT. Plates A3, A4, B3 and B4 are stained by NBC, INT and MTT. Picture A represents 0 minutes of incubation; picture B is taken after 120 minutes of incubation. This picture shows that colony staining can be done rapidly. In this case staining takes 2 hours or less at room temperature or 30-40 minutes at 35-40° C.

FIG. 5 illustrates different sizes and colors of different species. FIG. 5A is a colony of Bacillus brevis; E. coli O157:H7 in FIGS. 5B and C is Staphylococcus aureus. All species have been grown on one plate at the same time and temperature. Bacillus brevis reveals large, well stained mat colonies with inclusions in the middle. E. coli O157:H7 reveals brilliant medium size colonies and Staphylococcus aureus reveals small brilliant colonies with an easily visible dark ring encircling the mass. This experiment shows that colonies of different species can be stained rapidly and reveal different features useful to identify them. Incubation time of the plate shown was 18 hours. The nutrient medium was TSA. Chromogenic substrates mixture: TBC at 10 mg/ml, INT at 10 mg/ml and MIT at 0.5 mg/ml with Phosphoric Buffer Solution (PBS) pH7.6. The drop volume was 1.0 ml.

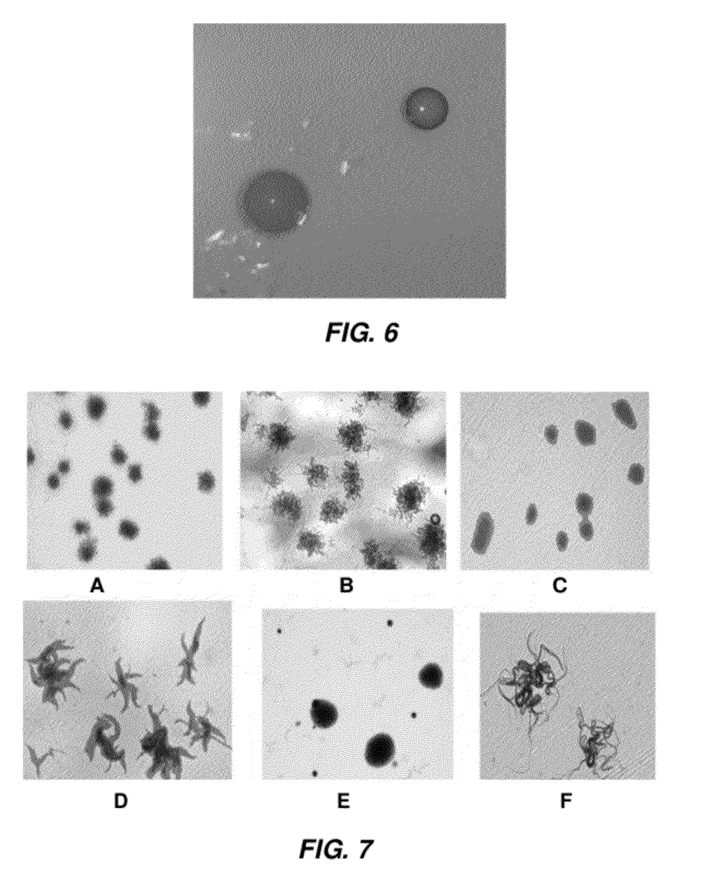

FIG. 6 demonstrates differences in color and shape of E. coli O157:H7 and Staphylococcus aureus grown on the same nutrient medium (TSA). Mixture of NBC (10 mg/ml), INT (10 mg/ml) and MTT (0.5 mg/ml) applied to lower side of nutrient agar is able to stain species differently: big red colonies E. coli O157:H7 and small violet with dark violet ring—Staphyllococcus aureus. This effect depends on features of the respiratory/dehydrogenase system of the different species. Thus, INT produces red formazan more intensively transformed by dehydrogenases of E. coli and less by Staphyllococcus aureus. MTT and NBC are responsible for a blue-violet color not visible in the black and white photographs.

Detection and differentiation of samples can be done 3-4 times faster, for example in 4-6 hours instead of 24 hours or 8-12 hours instead of 48 hours if microcolonies can be analyzed at an early stage of reproduction in the colony. Usually microcolonies are invisible using a microscope but staining by chromogen can make them visible even with the naked eye or low power magnification. The OBS method allows staining and analyzing of microcolonies in a rapid, simple, reliable and cost effective manner.

FIG. 7 shows microcolonies of several species stained with MTT. FIG. 7 A shows microcolonies of Entherobacter aeruginosa after 5 hours using MIT at 10 mg/ml PBS ph 7.6; FIG. 7 B shows Bacillus megatherium after 5 hours with MTT; FIG. 7 C shows E. coli O157 after 4 hours using MTT; FIG. 7 D shows Bacillus subtilis globiggii after 5 hours using MTT; FIG. 7 E shows a mixture of E. coli (large microcolonies), Staphylococcus aureus (small dark microcolonies) and Pseudumonas aeruginosa (small prolonged microcolonies) after 5 hours using MTT; FIG. 7F—Bacillus licheniformis after 5 hours using MTT. These results show that the OBS method is able to successfully stain microcolonies and reveal their specific shape.

The delivery time of substances to the colonies can differ. The bottom of nutrient agar surface can be shaped differently depending on the objectives sought. In one embodiment the growth medium's bottom surface is flat and at the same level as the ridge 6 of the container 2 as shown in FIG. 2.

In another embodiment the growth medium 5 can be made thinner inside the container 2. Thinning the growth medium 5 layer allows faster delivery through the growth medium. In this respect a thin layer is one that is made thinner than the customary growth medium layer, which is about 3 mm to about 6 mm thick, for the purpose of more rapid delivery of substances applied beneath the growth medium. Although such a thin layer might require additional support such as a polymer or plastic grid or, perhaps, a piece of multiply perforated cellophane with small holes as a substrate, more rapid delivery of substances to colonies or microcolonies can be accomplished in this way. Thus, if, for example, the incubation time through the relatively thicker layer is around one hour, the thinner layer might require only a quarter to half an hour or less.

The analytical and other substances must not always be dissolved in a liquid such as water, or a liquid buffer before application to the bottom surface. In some cases dry powder can also be applied to blotting paper or another carrier and then contacted to the bottom surface of the growth medium. In this embodiment solid diffusion can occur, or the powder could dissolve in liquid contained in the growth medium allowing molecules to pass through the growth layer and react with cells. However, in most cases to get the best substance transport through the growth medium, the substances applied to the bottom side of the medium should be able to dissolve in liquids present in the growth system.

As described above, the open bottom Petri plate enables the use of multiple new methods for laboratory based research and is even useful in the field. Further, it stimulates the creation of new methods of detection and identification for microorganisms. Among the new capabilities provided are: rapid detection of total viable organisms by one universal chromogenic substrate like MTT; use of mixtures of chromogenic substrates to reveal specific features of different species like Staph. aureus using a mixture of MTT, INT and NBC; rapid differentiation by morphology of microcolonies; detection by color changes of space surrounding colonies when pH sensitive dyes are used; detection and identification using fluorogenic substrates dissolved in growth medium; detection of antibiotic resistant microorganisms by addition of antibiotic to the bottom surface of the growth medium and staining by chromogen later when the size of colonies are different.

Several additional embodiments of the invention can be envisioned.

In one of these embodiments, rapid detection of total viable organisms, can be improved by taking a sample containing cells spread on the surface of a growth medium and incubated for 4 hours at optimal temperature. The removable membrane covering the open bottom Petri plate is peeled and the plate is placed on a 1.0 ml drop containing 15 mg of MIT in PBS, pH 7.6. The plate is returned to the incubator and incubated for 1 hour. During this time molecules of MTT pass through the growth medium and react with dehydrogenases of live cells. The cell bodies turn a dark violet color and become easily visible to the naked eye. Their specific morphology can be analyzed using the low magnification of a regular optical microscope as seen in FIG. 7. Thus, the enumeration of TVO can be done in 5 hours instead of 20-24 hours by the usual colony counting method. Practically all bacteria, actinomycetes, yeasts and many fungi can be analyzed using this method.

In another embodiment, a fluorescence related embodiment of the method, fluorogenic substrates can be used instead of MTT such as 4-methylumbelliferyl acetate, butyrate, phosphate or their mixtures. To avoid self fluorescence of nutrient media, microcolonies need to be removed by imprinting on a black non-fluorescent filtration membrane or paper.

Using another embodiment, rapid identification of Methicillin Resistant Staphylococcus Aureus (MRSA) can be accomplished by taking a sample containing antibiotic resistant MRSA, Vancomycin-Resistant Enterococcus (VRE) or other species spread on the surface of a nutrient agar and incubated 3 hours at 35° C. During this time the live bacteria form very small invisible microcolonies. After 3 hours of incubation the removable membrane is peeled from the bottom of the Petri plate, and the plate mounted on 1.0 ml drop of Oxacillin at 0.02 mg/ml concentration. Molecules of Oxacillin are spread on the nutrient agar to stop the growth of regular Staph. Aureus leaving the MRSA to continue growing. After an additional 7 hours of incubation, the plate is placed on a 1.0 ml drop of mixture containing MTT (0.5 mg), INT (10.0 mg) and NBC (10.0 mg). After 40 to 60 minutes of further incubation, the MRSA microcolonies can be identified by their now violet color featuring a blue ring around the microcolony. Thus, live MRSA can be identified in just 11 hours instead of the 48 hours required by the usual method or by CHROMagar™.

In yet another embodiment colonies can be differentiated by color or fluorescence. Differentiation and identification of colonies and microcolonies can be made by using chromogenic or fluorogenic mixtures. One of those mixtures can be NBC (10 mg), INT (10 mg) and MTT (0.5 mg) dissolved in one milliliter of PBS pH 7.6 (see FIGS. 5 and 6). Colonies of different species can be recognized by shape, size, color and features the ring around colonies of Staph. aureus. A sample is spread on the surface of growth medium containing plate and incubated 18-24 hours. A mixture of tetrazolium salts is added to the bottom surface. The resulting staining effect appears after 40 to 60 minutes of incubation.

Reference has been made in detail to embodiments of the invention. Examples of the embodiments are illustrated in the accompanying figures. While the invention has been described in conjunction with the embodiments discussed herein, it is to be understood that the invention is not limited to the embodiments discussed. Rather, the invention is intended to cover alternatives, modifications and equivalents, which may be included within the spirit and scope of the invention, as defined by the appended claims. Additionally, in the detailed description of the present specification, numerous specific details are set forth to provide a thorough understanding of the present disclosure. However, it will be apparent to one of ordinary skill in the art that the present invention may be practiced without these specific details. In other instances, well-known methods, procedures, components, and microbiological details have not been described in detail so as not to unnecessarily obscure aspects of the present disclosure.

1. An apparatus for growing microorganisms comprising:
a container having at least one wall, a substantially open top side and a substantially open bottom side;
a top cover for covering the open top side to protect the microorganisms disposed on a microbiological growth medium;
a structure for supporting the growth medium; and
a removable bottom cover for allowing access to the bottom side of the growth medium,
access to the bottom side of the growth medium allowing analytical substances to be placed in contact with the growth medium so the substances can pass through the growth medium and interact with the microorganisms disposed on the opposite side of the growth medium.

2. The apparatus of claim 1 wherein the bottom cover is a removable membrane.

3. The apparatus of claim 1 wherein a top cover is not used.

4. The apparatus of claim 1 wherein a bottom cover is not used.

5. The apparatus of claim 1 wherein the container supporting the growth medium has structures that prevent slippage and damage of the growth medium.

6. The apparatus of claim 1 wherein the container containing the growth medium comprises structures for supporting the growth medium.

7. The apparatus of claim 1 wherein the removable bottom cover is coated with adhesive to hold the cover in place.

8. The apparatus of claim 1 wherein the removable bottom cover is attached to the container using threads.

9. The apparatus of claim 1 wherein the removable bottom cover is attached to the container by using a friction fit connection.

10. The apparatus of claim 1 wherein the removable bottom cover is attached to the bottom of the container using a mechanical connector.

11. The apparatus of claim 1 wherein the container comprises a structure spanning the substantially open bottom side for supporting the growth medium while allowing access through the structure to the bottom of the growth medium.

12. A method for detecting and identifying microorganisms comprising the following steps:
preparing a container for growing the microorganisms having at least one wall, a substantially open top side, a substantially open bottom side, a removable top cover, a microbiological growth medium, a structure for supporting the microbiological growth medium, and a removable bottom cover;
growing colonies of microorganisms on the top surface of the microbiological growth medium;
removing the bottom cover;
placing the bottom surface of the growth medium in transportable communication with a substance capable of passing through the growth medium;
incubating to allow molecules of the substance to reach the microorganisms and react with them; and
detecting and analyzing results of the reaction between the microorganisms and the substance.

13. The method of claim 12 where the substance comprises a liquid.

14. The method of claim 12 where the substance comprises a dry powder.

15. The method of claim 12 where blotting paper containing the substance is placed in transportable communication with growth medium through the bottom side of the container;

16. The method of claim 12 where the microorganisms growing on the top surface of the growth medium comprise at least one of the following: bacteria, fungi, actinomycetes, molds, eukaryotic cells, animal cells, and plant cells.

17. The method of claim 12 where the microbiological growth medium contains a gelling substance for solidifying the microbiological growth medium.

18. The method of claim 12 wherein the substance capable of passing through the growth medium comprises at least one of the following: a chromogenic substrate, a fluorogenic substrate, a mixture of chromogenic and fluorogenic substrates, an antibiotic, a biochemical dye, a pH indicator, a growth accelerator, a growth decelerator, antibodies, and an enzyme.

19. A method for detecting and identifying microorganisms comprising the following steps:
preparing a container for growing the microorganisms having at least one wall, a substantially open top side, a substantially open bottom side, a removable top cover, a microbiological growth medium, a structure for supporting the microbiological growth medium, and a removable bottom cover;
growing colonies of microorganisms on the top surface of the microbiological growth medium;
dispensing a substance capable of reacting with the microorganisms over the microorganisms on the top side of the growth medium;
removing the bottom cover;
placing a material in drawable communication with the bottom side of the growth medium through the substantially open bottom side of the container for drawing the substance from the top side of the growth medium to the material;
incubating to allow the substance to interact with the microorganisms and be drawn to the material; and
detecting and analyzing results of the interaction between the microorganisms and the substance.

20. The method of claim 19 where the material is blotting paper.
